(19) United States
(12) Patent Application Publication
(10) Pub. No.: US 2010/0330029 A1
(43) Pub. Date: Dec. 30, 2010

(54) Cancer Treatments with Radiation and Immunocytokines

(75) Inventors: Thomas Wickham, Groton, MA (US); Ulrike Gnad-Vogt, Frankfurt (DE); Sylvia Holden, Woburn, MA (US); Stephan G. Klinz, Norwood, MA (US); Karl-Josef Kallen, Gross-Gerau (DE)

Correspondence Address: GOODWIN PROCTER LLP;PATENT ADMINISTRATOR, 53 STATE STREET, EXCHANGE PLACE, BOSTON, MA, 02109-2881, US

(73) Assignee: Merck Patent GmbH, Darmstadt (DE)

(21) Appl. No.: 12/603,363

(22) Filed: Oct. 21, 2009

Related U.S. Application Data
(60) Provisional application No. 61/107,146, filed on Oct. 21, 2008.

(51) Int. Cl. A61K 38/20; A61P 35/00; A61P 37/04

(52) U.S. Cl. 424/85.2

(57) ABSTRACT

The present invention is directed to a method for treating tumors and cancer cells by administering an immunocytokine following radiation treatment. This combination of treatments can stimulate an immune response at irradiated and non-irradiated sites, which is useful in eradicating cancer cells that have spread from the site of the primary tumor. In addition, immunocytokines can be administered at a dose that is less that the maximum tolerated dose, which reduces the side effects associated with immunocytokine therapy.

### REFERENCE TO RELATED APPLICATIONS

This application claims the benefit of and priority to U.S. Provisional Patent Application No. 61/107,146, filed Oct. 21, 2008, the complete disclosure of which is incorporated by reference herein.

### BACKGROUND OF THE INVENTION

Effective treatment of diseases such as cancer requires robust immune responses by one or more effector cell types such as natural killer (NK) cells, macrophages and T lymphocytes. However, existing cancer therapies, for example, radiation treatment and chemotherapy, target rapidly dividing cells, and therefore actually destroy immune cells. In addition, the tumor environment itself is immunosuppressive.

To combat the immunosuppressive effects of current cancer therapies, studies have been performed to investigate the administration of the cytokine IL-2 in combination with radiation therapy. See Jacobs et al. (2005), Cancer Immunol. Immunother., 54:792-798; Everse et al. (1997), Int. J. Cancer, 72:1003-1007; Lam, et al. (1995), Journal of Immunotherapy, 18 (1):28-34; and Jürgenliemk-Schulz et al., (1997), Radiation Oncology Investigations, 5:54-61. While some response was seen for certain cancer types, in many cases tumors were not completely eradicated. In other cases, tumors would recur. In addition, certain types of cancer showed no improvement with treatment. Further, IL-2 is known to produce serious side effects, including vascular leakage syndrome, in which fluid leaks out of blood vessels causing low blood pressure, difficulty breathing, and edema. Therefore, improvements are needed that increase the stimulation of systemic immune responses while at the same time minimizing side effects produced by the therapies.

### SUMMARY OF THE INVENTION

The present invention relates to methods for reducing tumor or cancer cell growth in a mammal. The methods reduce tumor or cancer cell growth by following irradiation of a tumor with administration of an immunocytokine, to enhance an immune response which facilitates the reduction in growth of the tumor. Some methods of practicing the invention may reduce tumor size, inhibit metastasis, inhibit tumor regrowth, inhibit relapse, increase average time to progression, increase average survival time, or promote partial or complete responses to a therapeutic regimen, which may include additional therapeutic agents or activities, such as surgery. The invention further relates to healthcare business methods for authorizing administration of, or authorizing payment for the administration of, the immunocytokines of the present invention.

The present invention also relates to uses of immunocytokines to reduce tumor or cancer cell growth by following irradiation of a tumor with use of an immunocytokine, to enhance an immune response which facilitates the reduction in growth of the tumor. Some uses of immunocytokines in accordance with the invention may reduce tumor size, inhibit metastasis, inhibit tumor regrowth, inhibit relapse, increase average time to progression, increase average survival time, or promote partial or complete responses to a therapeutic regimen, which may include additional therapeutic agents or activities, such as surgery.

In one aspect, the invention relates to a method of reducing tumor or cancer cell growth by administering an immunocytokine to a mammal (e.g. a human) who has a tumor that has already been irradiated. In another aspect, the invention relates to a method of enhancing a systemic immune response in a mammal having cancer cells at multiple locations, including administering an immunocytokine after a subset of the locations have been irradiated. The irradiation enhances an immune response both at irradiated and non-irradiated locations.

In another aspect, the invention relates to the use of an immunocytokine to reduce tumor or cancer cell growth in a mammal (e.g. a human) who has a tumor that has already been irradiated. In another aspect, the invention relates to the use of an immunocytokine to enhance a systemic immune response in a mammal having cancer cells at multiple locations, including use of an immunocytokine after a subset of the locations have been irradiated. The irradiation enhances an immune response both at irradiated and non-irradiated locations.

In another aspect, the present invention relates to a healthcare method that includes authorizing the administration of, or authorizing payment for the administration of, an immunocytokine to a mammal with a tumor or cancer cells that were previously irradiated.

In some embodiments of the invention, the dose of radiation (e.g. gamma radiation) given was at least 1, at least 2, or at least 3 Gy per day. In certain embodiments, the dose of radiation given is 1-4 Gy, 1-10 Gy, 1-20 Gy, 2-4 Gy, 2-10 Gy, 2-20 Gy, 3-4 Gy, 3-10 Gy, or 3-20 Gy per day.

One advantage to combining radiation and immunocytokine treatment according to the methods and uses of the present invention is that a lower dose of immunocytokine can be administered, reducing the possibility of side effects. In one embodiment of the invention, the dose of immunocytokine is administered or used at a dose less than the maximum tolerated dose of the immunocytokine. In another embodiment, the immunocytokine is administered or used at a dose less than half, less than a third, less than a quarter, or less than one-tenth of the maximum tolerated dose. In another embodiment, the immunocytokine includes interleukin-2, optionally incorporating one or more mutations such as a D20T mutation. In yet another embodiment, the immunocytokine includes interleukin-12.

The number of radiation treatments and the amount of time between the radiation treatment and the administration of the immunocytokine can vary according to the methods or uses of the invention. In some embodiments, the tumor is irradiated on only one day, or is irradiated on multiple days over a period that does not exceed 14 days. In certain embodiments, the period is 6-8 days, 4-10 days, 2-12 days or 1-14 days. In particular embodiments, the immunocytokine is administered at least two days, four days or six days after the end of the period. In another embodiment, the immunocytokine is administered within 21 days, 18 days, 15 days, 12 days or 8 days of an initial administration of radiation to the tumor. In one embodiment, the immunocytokine is administered at least five days, seven days, nine days or 12 days after an initial administration of radiation to the tumor.

Embodiments of the invention may include combining known cancer treatment methods with the methods of the invention. In some embodiments, the method further includes surgically removing at least a portion of the tumor, administering an additional therapeutic agent, or irradiating the tumor.

Other embodiments of the invention may include using immunocytokines in combination with known cancer treatments. In some embodiments, immunocytokines may be used in a mammal having had at least a portion of a tumor removed, with a previously irradiated tumor, or in combination with use of an additional therapeutic agent.

Some methods of practicing and uses of the invention may combine two or more of the conditions defined above. For example, in one embodiment, the radiation given is at least 1 Gy/day (e.g. at least 2, at least 3, 1-4, 1-10, 1-20, 2-4, 2-10, 2-20, 3-4, 3-10, or 3-20 Gy/day), to only a subset of locations of cancer cells in the mammal. The immunocytokine may be administered or used within 21 days (e.g. within 18, within 15, within 12, or within 8 days) of an initial administration of radiation and may be administered or used at a dose less than half (e.g. less than a third, less than a quarter, or less than one-tenth) of the maximum tolerated dose. In another embodiment, the radiation given is 1 Gy/day (e.g. at least 2, at least 3, 1-4, 1-10, 1-20, 2-4, 2-10, 2-20, 3-4, 3-10, or 3-20 Gy/day) and the immunocytokine is administered or used within 21 days (e.g. within 18, within 15, within 12, or within 8 days) of an initial administration of radiation, optionally at a dose less than half (e.g. less than a third, less than a quarter, or less than one-tenth) of the maximum tolerated dose. In another embodiment, the radiation given is at least one 1 Gy/day (e.g. at least 2, at least 3, 1-4, 1-10, 1-20, 2-4, 2-10, 2-20, 3-4, 3-10, or 3-20 Gy/day) and the immunocytokine is administered or used at a dose less than half of the maximum tolerated dose. In another embodiment, the radiation is administered to only a subset of locations of cancer cells in the mammal and the immunocytokine is administered or used within 21 days (e.g. within 18, within 15, within 12, or within 8 days) of an initial administration of radiation, optionally at a dose less than half (e.g. less than a third, less than a quarter, or less than one-tenth) of the maximum tolerated dose. In another embodiment, the radiation is administered to only a subset of locations of cancer cells in the mammal and the immunocytokine is administered or used at a dose less than half (e.g. less than a third, less than a quarter, or less than one-tenth) of the maximum tolerated dose. In another embodiment, the immunocytokine is administered to or used in a mammal with a previously irradiated tumor within 21 days (e.g. within 18, within 15, within 12, or within 8 days) of an initial administration of radiation and the immunocytokine is administered at a dose less than half (e.g. less than a third, less than a quarter, or less than one-tenth) of the maximum tolerated dose.

### BRIEF DESCRIPTION OF THE DRAWINGS

FIG. 1 depicts an exemplary dosing schedule wherein the radiation “R” and the immunocytokine “IC” can each be administered as single doses, separated by an interval 1-1.

FIG. 2 depicts an exemplary dosing schedule wherein the radiation “R” is administered on only one day, followed by the administration of an immunocytokine “IC” on multiple days.

FIG. 3 depicts an exemplary dosing schedule wherein the radiation “R” is administered on more than one day, followed by the administration of an immunocytokine “IC” on only one day “IC1.”

FIG. 4 depicts an exemplary dosing schedule wherein the radiation “R” can be administered on more than one day, followed by administration of an immunocytokine “IC” on more than one day.

FIG. 5 depicts an exemplary dosing schedule wherein the radiation “R” can be administered on more than one day, followed by administration of an immunocytokine “IC” on more than one day, and the start “IC1” of administration of the immunocytokine can occur before the end “R2” of the period of irradiation.

FIG. 6 depicts tumor size over time in animals treated with chemoradiation plus NHS-muIL12 (open squares), chemoradiation plus EMD521873 (open circles), chemoradiation alone (closed triangles), NHS-muIL12 alone (closed squares), EMD521873 alone (closed circles), or control treatment (X).

FIG. 7 depicts tumor size over time in animals treated with the immunocytokine NHS-IL12 in combination with chemoradiation (closed circles), EMD521873 (open circles) in combination with chemoradiation, no treatment (X), chemoradiation alone (open squares), EMD521873 alone (open diamonds) or NHS-muIL12 alone (open triangles).

FIG. 8 depicts tumor size over time in animals treated with chemoradiation plus EMD521873 (open circles), chemoradiation alone (open triangles), control treatment (X) or EMD521873 alone (open boxes).

FIG. 9 depicts tumor size over time in animals treated with 1 mg/kg EMD521873 plus chemoradiation (closed square), 5 mg/kg EMD521873 plus chemoradiation (closed triangle) and 15 mg/kg EMD521873 plus chemoradiation (closed circle), 1 mg/kg EMD 521873 alone (open square), 5 mg/kg EMD521873 alone (open triangle) and 15 mg/kg EMD521873 alone (open circle), chemoradiation alone (+), or vehicle alone (X).

### DETAILED DESCRIPTION OF THE INVENTION

The methods of present invention provide for a combination therapy for a mammal with one or more tumors, which includes radiation and immunocytokine administration, and results in an increased immune response and subsequent reduction in tumor growth. Methods of the present invention are useful for treating individual tumors, multiple tumors (including non-irradiated tumors) or metastases because a systemic immune response is activated. Following radiation treatment, immunocytokines can be administered at a dose lower than the maximum tolerated dose, which is advantageous because using a lower dose of immunocytokines can lead to fewer side effects.

### Radiation Therapy

According to the methods of the present invention, radiation can be administered in combination with immunocytokines to treat cancer. Radiation therapy typically uses a beam of high-energy particles or waves, such as X-rays and gamma rays, to eradicate cancer cells by inducing mutations in cellular DNA. Cancer cells divide more rapidly than normal cells, making tumor tissue more susceptible to radiation than normal tissue. Any type of radiation can be administered to a patient, so long as the dose of radiation is tolerated by the patient without significant negative side effects. Suitable types of radiotherapy include, for example, ionizing radiation (e.g., X-rays, gamma rays, or high linear energy radiation). Ionizing radiation is defined as radiation comprising particles or photons that have sufficient energy to produce ionization, i.e., gain or loss of electrons (as described in, for example, U.S. Pat. No. 5,770,581). The effects of radiation can be at least partially controlled by the clinician. The dose of radiation is preferably fractionated for maximal target cell exposure and reduced toxicity. Radiation can be administered concurrently with radiosensitizers that enhance the killing of tumor cells, or with radioprotectors (e.g., IL-1 or IL-6) that protect healthy tissue from the harmful effects of radiation. Similarly, the application of heat, i.e., hyperthermia, or chemotherapy can sensitize tissue to radiation.

The source of radiation can be external or internal to the patient. External radiation therapy is most common and typically involves directing a beam of high-energy radiation (a particle beam) to a tumor site through the skin using, for instance, a linear accelerator. While the beam of radiation is localized to the tumor site, it is nearly impossible to avoid exposure of normal, healthy tissue. However, external radiation is usually well tolerated by patients.

In another example, radiation is supplied externally to a patient using gamma rays. Gamma rays are produced by the breakdown of radioisotopes such as cobalt 60. Using a treatment called the “Gamma Knife®,” gamma rays can be tightly focused to target tumor tissue only, such that very little healthy tissue is damaged. On the other hand, X-rays, produced by a particle accelerator, can be used to administer radiation over a larger area of the body.

Internal radiation therapy involves implanting a radiation-emitting source, such as beads, wires, pellets, capsules, etc., inside the body at or near the tumor site. The radiation used comes from radioisotopes such as, but not limited to, iodine, strontium, phosphorus, palladium, cesium, iridium, phosphate or cobalt. Such implants can be removed following treatment, or left in the body inactive. Types of internal radiation therapy include, but are not limited to, brachytherapy, interstitial irradiation, and intracavity irradiation. A currently less common form of internal radiation therapy involves biological carriers of radioisotopes, such as with radioimmunotherapy wherein tumor-specific antibodies bound to radioactive material are administered to a patient. The antibodies bind tumor antigens, thereby effectively administering a dose of radiation to the relevant tissue.

Radiation therapy is useful as a component of a regimen to control the growth of a primary tumor (see, e.g. Comphausen et al. (2001) “Radiation Therapy to a Primary Tumor Accelerates Metastatic Growth in Mice,” Cancer Res. 61:2207-2211). Although radiation therapy alone may be less effective at destroying or preventing metastases, combining radiation with an immunocytokine as described herein can enhance the local and systemic efficacy of radiation therapy. According to the methods of the present invention, radiation can be administered to a subset of tumors or cancer cells in an mammal with multiple tumors or cancer cells. In one embodiment, the subset of tumors is one tumor. After a subset of the locations have been irradiated, an immunocytokine is administered. The irradiation enhances an immune response at irradiated and non-irradiated locations, compared to immunocytokine administration alone. Thus, the methods of the present invention may be effective in treating mammals in which cancer cells have spread from one or more tumors to other locations in the body.

Because radiation kills immune effector cells, the dose and timing of the radiation is important. T cells and dendritic cells in an irradiated tumor decrease immediately after irradiation; however, T-cell levels rebound higher than baseline levels. No matter the method of administration, a complete dose of radiation can be administered over the course of one day. Preferably, the total dose is fractionated and administered over several days. Accordingly, a daily dose of radiation will comprise approximately 3-20 Gy/day, for example, at least 2, at least 3, 1-4, 1-10, 1-20, 2-4, 2-10, 2-20, 3-4, 3-10, 3-20 Gy/day. The daily dose can be administered as a single dose, or can be a “microfractionated” dose administered in two or more portions over the course of a day. When internal sources of radiation are employed, e.g., brachytherapy or radio-immunotherapy, the exposure time typically will increase, with a corresponding decrease in the intensity of radiation.

As used herein, an “initial administration of radiation” can be a single dose, or the beginning of a series of irradiations spread over several days. While the mammal may have previously received one or more courses of radiation therapy, an “initial administration” is separated in time from any preceding courses of radiation. For example, if a mammal receives radiation on a Monday, Tuesday, and Wednesday, the Tuesday administration would not be an “initial administration.” Similarly, if a mammal receives radiation every Monday, Wednesday, and Friday for three weeks, the first Monday may be an “initial administration,” but the second and third Mondays would not be. Generally, an initial administration of radiation is preceded by seven or more days without receiving radiation (e.g. without receiving a therapeutic dose of radiation, such as a dose of at least 1 Gy).

A “period” of irradiation, as used herein, has one initial administration of radiation followed by administrations of radiation on one or more additional days. The additional administrations of radiation may be on consecutive days (e.g. Monday, Tuesday, Wednesday), alternate days (e.g. Monday, Wednesday, Friday), etc., or 2 or 3 or 4 or 5 or 6 times per week, for example. A given period of radiation would not include any seven consecutive radiation-free days. Rather, resumption of regular administration of radiation after a seven day hiatus would generally mark the beginning of a new “period.”

### Immunocytokines

As used herein, the term “immunocytokine” is understood to mean a fusion of (i) an antibody binding domain having binding specificity for, and capable of binding a pre-selected antigen, for example, a cell-type specific antigen, and (ii) a cytokine that is capable of inducing or stimulating a cytocidal immune response typically against a cancer cell. Examples of pre-selected antigens include cell surface antigens such as on cancer cells, and other antigens that are characteristic of the tumor microenvironment, whether or not directly associated with a cell, such as antigens that may be secreted or otherwise released or deposited in the vicinity of a tumor; antigens associate with the extracellular membrane in the vicinity of a tumor; or antigens associated with non-malignant cells that are in contact with and/or infiltrating the tumor. Preferred antigens are target antigens that are characteristic of tumor cells, such as tumor specific antigens. Accordingly, the immunocytokine is capable of selectively delivering the cytokine to a target (which typically is a cell) in vivo so that the cytokine can mediate a localized immune response against a target cell. For example, if the antibody component of the immunocytokine selectively binds an antigen on a cancer cell, such as a cancer cell in a solid tumor, and in particular a larger solid tumor of greater than about 100 mm³, the immunocytokine exerts localized anti-cancer activity.

As used herein, the term “antibody binding domain” is understood to mean at least a portion of an immunoglobulin heavy chain, for example, an immunoglobulin variable region capable of binding a pre-selected antigen such as a cell type. The antibody binding domain also preferably comprises at least a portion of an immunoglobulin constant region including, for example, a CH1 domain, a CH2 domain, and a CH3 domain, or at least a CH3 domain, or one or more portions thereof. Furthermore, the immunoglobulin heavy chain may be associated, either covalently or non-covalently, to an immunoglobulin light chain comprising, for example, an immunoglobulin light chain variable region and optionally light chain constant region. Accordingly, it is contemplated that the antibody binding domain may comprise an intact antibody or a fragment thereof, or a single chain antibody, capable of binding the preselected antigen.

With regard to the immunocytokine, it is contemplated that the antibody fragment may be linked to the cytokine by a variety of ways well known to those of ordinary skill in the art. For example, the antibody binding site preferably is linked via a polypeptide bond or linker to the cytokine in a fusion protein construct. Alternatively, the antibody binding site may be chemically coupled to the cytokine via reactive groups, for example, sulfhydryl groups, within amino acid side chains present within the antibody binding site and the cytokine.

As used herein, the term “cytokine” is understood to mean any protein or peptide, analog or functional fragment thereof, which is capable of stimulating or inducing a cytocidal immune response against a preselected cell-type, for example, a cancer cell or a virally-infected cell, in a mammal. Accordingly, it is contemplated that a variety of cytokines can be incorporated into the immunocytokines of the invention. Cytokines that can be incorporated into the immunocytokines of the invention include, for example, tumor necrosis factors, interleukins, colony stimulating factors, and lymphokines, as well as others known in the art. Preferred tumor necrosis factors include, for example, tissue necrosis factor α (TNF α). Preferred interleukins include, for example, interleukin-2 (IL-2), interleukin-4 (IL-4), interleukin-5 (IL-5), interleukin-7 (IL-7), interleukin-12 (IL-12), interleukin-15 (IL-15) and interleukin-18 (IL-18). Preferred colony stimulating factors include, for example, granulocyte-macrophage colony stimulating factor (GM-CSF) and macrophage colony stimulation factor (M-CSF). Preferred lymphokines include, for example, lymphotoxin (LT). Other useful cytokines include interferons, including IFN-α, IFN-β and IFN-γ, all of which have immunological effects, as well as anti-angiogenic effects, that are independent of their anti-viral activities. Altered forms of cytokines may also be used. For example, mutations in the cytokine portion of the immunocytokine may impart improved properties to the immunocytokine, such as the D20T mutation of IL-2 which increases the selectivity of the altered IL-2 to its high-affinity receptor relative to wild-type IL-2, thus decreasing its toxicity, as disclosed in U.S. Pat. No. 7,186,804.

The gene encoding a particular cytokine of interest can be cloned de novo, obtained from an available source, or synthesized by standard DNA synthesis from a known nucleotide sequence. For example, the DNA sequence of LT is known (see, for example, Nedwin et al. (1985) Nucleic Acids Res. 13: 6361), as are the sequences for IL-2 (see, for example, Taniguchi et al. (1983) Nature 302: 305-318), GM-CSF (see, for example, Gasson et al. (1984) Science 266: 1339-1342), and TNFα (see, for example, Nedwin et al. (1985) Nucleic Acids Res. 13: 6361).

In a preferred embodiment, the immunocytokines are recombinant fusion proteins produced by conventional recombinant DNA methodologies, i.e., by forming a nucleic acid construct encoding the chimeric immunocytokine. The construction of recombinant antibody-cytokine fusion proteins has been described in the prior art. See, for example, Gillies et al. (1992) Proc. Natl. Acad. Sci. USA 89: 1428-1432; Gillies et al. (1998) J. Immunol. 160: 6195-6203; and U.S. Pat. No. 5,650,150. Preferably, a gene construct encoding the immunocytokine of the invention includes, in 5′ to 3′ orientation, a DNA segment encoding an immunoglobulin heavy chain variable region domain, a DNA segment encoding an immunoglobulin heavy chain constant region, and a DNA encoding the cytokine. The fused gene is assembled in or inserted into an expression vector for transfection into an appropriate recipient cell where the fused gene is expressed. The hybrid polypeptide chain preferably is combined with an immunoglobulin light chain such that the immunoglobulin variable region of the heavy chain (V_{H}) and the immunoglobulin variable region of the light chain (V_{L}) combine to produce a single and complete site for binding a preselected antigen. In a preferred embodiment, the immunoglobulin heavy and light chains are covalently coupled, for example, by means of an interchain disulfide bond. Furthermore, two immunoglobulin heavy chains, either one or both of which are fused to a cytokine, can be covalently coupled, for example, by means of one or more interchain disulfide bonds.

Accordingly, methods of the invention are useful to enhance the anti-tumor activity of an immunocytokine used in a therapeutic method to treat a tumor, including immunocytokine compositions and methods disclosed in WO99/29732, WO99/43713, WO99/52562, WO99/53958, and WO01/10912, and antibody-based fusion proteins with an altered amino acid sequence in the junction region.

Immunocytokines of the invention may be considered chimeric by virtue of two aspects of their structure. First, the immunocytokine is chimeric in that it includes an immunoglobulin heavy chain having antigen binding specificity linked to a given cytokine. Second, an immunocytokine of the invention may be chimeric in the sense that it includes an immunoglobulin variable region (V) and an immunoglobulin constant region (C), both of which are derived from different antibodies such that the resulting protein is a V/C chimera. For example, the variable and constant regions may be derived from naturally occurring antibody molecules isolatable from different species. See, for example, U.S. Pat. No. 4,816,567. Also embraced are constructs in which either or both of the immunoglobulin variable regions comprise framework region (FR) sequences and complementarity determining region (CDR) sequences derived from different species. Such constructs are disclosed, for example, in Jones et al. (1986) Nature 321: 522-525, Verhoyen et al. (1988) Science 239: 1534-1535, and U.S. Pat. Nos. 5,225,539 and 5,585,089. Furthermore, it is contemplated that the variable region sequences may be derived by screening libraries, for example, phage display libraries, for variable region sequences that bind a preselected antigen with a desired affinity. Methods for making and screening phage display libraries are disclosed, for example, in Huse et al. (1989) Science 246: 1275-1281 and Kang et al. (1991) Proc. Natl. Acad. Sci. USA 88: 11120-11123.

The immunoglobulin heavy chain constant region domains of the immunocytokines can be selected from any of the five immunoglobulin classes referred to as IgA (Igα), IgD (Igδ), IgE (Igε), IgG (Igγ), and IgM (Igμ). However, immunoglobulin heavy chain constant regions from the IgG class are preferred. Furthermore, it is contemplated that the immunoglobulin heavy chains may be derived from any of the IgG antibody subclasses referred to in the art as IgG1, IgG2, IgG3 and IgG4. As is known, each immunoglobulin heavy chain constant region comprises four or five domains. The domains are named sequentially as follows: CH1-hinge-CH2-CH3-(—CH4). CH4 is present in IgM, which has no hinge region. The DNA sequences of the heavy chain domains have cross homology among the immunoglobulin classes, for example, the CH2 domain of IgG is homologous to the CH2 domain of IgA and IgD, and to the CH3 domain of IgM and IgE. The immunoglobulin light chains can have either a kappa (κ) or lambda (λ) constant chain. Sequences and sequence alignments of these immunoglobulin regions are well known in the art (see, for example, Kabat et al., “Sequences of Proteins of Immunological Interest,” U.S. Department of Health and Human Services, third edition 1983, fourth edition 1987, and Huck et al. (1986) Nuc. Acids Res. 14: 1779-1789).

In preferred embodiments, the variable region is derived from an antibody specific for a preselected cell surface antigen (an antigen associated with a diseased cell such as a cancer cell or virally-infected cell), and the constant region includes CH1, and CH2 and CH3 domains from an antibody that is the same or different from the antibody that is the source of the variable region. In the practice of this invention, the antibody portion of the immunocytokine preferably is non-immunogenic or is weakly immunogenic in the intended recipient. Accordingly, the antibody portion, as much as possible, preferably is derived from the same species as the intended recipient. For example, if the immunocytokine is to be administered to humans, the constant region domains preferably are of human origin. See, for example, U.S. Pat. No. 4,816,567. Furthermore, when the immunoglobulin variable region is derived from a species other than the intended recipient, for example, when the variable region sequences are of murine origin and the intended recipient is a human, then the variable region preferably comprises human FR sequences with murine CDR sequences interposed between the FR sequences to produce a chimeric variable region that has binding specificity for a preselected antigen but yet while minimizing immunoreactivity in the intended host. The design and synthesis of such chimeric variable regions are disclosed in Jones et al. (1986) Nature 321: 522-525, Verhoyen et al. (1988) Science 239: 1534-1535, and U.S. Pat. Nos. 5,225,539 and 5,585,089. The cloning and expression of a humanized antibody-cytokine fusion protein, KS-1/4 anti-EpCAM antibody-IL-12 fusion protein, as well as its ability to eradicate established colon carcinoma metastases has been described in Gillies et al. (1998) J. Immunol. 160: 6195-6203.

The gene encoding the cytokine is joined, either directly or by means of a linker, for example, by means of DNA encoding a (Gly₄-Ser)₃ linker in frame to the 3′ end of the gene encoding the immunoglobulin constant region (e.g., a CH2 or CH3 exon). In certain embodiments, the linker can comprise a nucleotide sequence encoding a proteolytic cleavage site. This site, when interposed between the immunoglobulin constant region and the cytokine, can be designed to provide for proteolytic release of the cytokine at the target site. For example, it is well known that plasmin and trypsin cleave after lysine and arginine residues at sites that are accessible to the proteases. Many other site-specific endoproteases and the amino acid sequences they cleave are well-known in the art. Preferred proteolytic cleavage sites and proteolytic enzymes that are reactive with such cleavage sites are disclosed in U.S. Pat. Nos. 5,541,087 and 5,726,044.

The nucleic acid construct optionally can include the endogenous promoter and enhancer for the variable region-encoding gene to regulate expression of the chimeric immunoglobulin chain. For example, the variable region encoding genes can be obtained as DNA fragments comprising the leader peptide, the VJ gene (functionally rearranged variable (V) regions with joining (J) segment) for the light chain, or VDJ gene for the heavy chain, and the endogenous promoter and enhancer for these genes. Alternatively, the gene encoding the variable region can be obtained apart from endogenous regulatory elements and used in an expression vector which provides these elements.

Variable region genes can be obtained by standard DNA cloning procedures from cells that produce the desired antibody. Screening of the genomic library for a specific functionally rearranged variable region can be accomplished with the use of appropriate DNA probes such as DNA segments containing the J region DNA sequence and sequences downstream. Identification and confirmation of correct clones is achieved by sequencing the cloned genes and comparison of the sequence to the corresponding sequence of the full length, properly spliced mRNA.

The target antigen can be a cell surface antigen of a tumor cell, a cancer cell, or other neoplastic cell. Genes encoding appropriate variable regions can be obtained generally from immunoglobulin-producing lymphoid cell lines. For example, hybridoma cell lines producing immunoglobulin specific for tumor associated antigens or viral antigens can be produced by standard somatic cell hybridization techniques well known in the art (see, for example, U.S. Pat. No. 4,196,265). These immunoglobulin producing cell lines provide the source of variable region genes in functionally rearranged form. Alternatively, variable region sequences may be derived by screening libraries, for example, phage display libraries, for variable region sequences that bind a preselected antigen with a desired affinity. Methods for making and screening phage display libraries are disclosed, for example, in Huse et al. (1989) Science 246: 1275-1281 and Kang et al. (1991) Proc. Natl. Acad. Sci. USA 88: 11120-11123.

The DNA fragment encoding the functionally active variable region gene is linked to a DNA fragment containing the gene encoding the desired constant region (or a portion thereof). Immunoglobulin constant regions (heavy and light chain) can be obtained from antibody-producing cells by standard gene cloning techniques. Genes for the two classes of human light chains (κ and λ) and the five classes of human heavy chains (α, δ, ε, γ and μ) have been cloned, and thus, constant regions of human origin are readily available from these clones.

The fused gene encoding the hybrid immunoglobulin heavy chain is assembled or inserted into an expression vector for incorporation into a recipient cell. The introduction of the gene construct into plasmid vectors can be accomplished by standard gene splicing procedures. The chimeric immunoglobulin heavy chain can be co-expressed in the same cell with a corresponding immunoglobulin light chain so that a complete immunoglobulin can be expressed and assembled simultaneously. For this purpose, the heavy and light chain constructs can be placed in the same or separate vectors.

Recipient cell lines are generally lymphoid cells. The preferred recipient cell is a myeloma (or hybridoma). Myelomas can synthesize, assemble, and secrete immunoglobulins encoded by transfected genes and they can glycosylate proteins. Exemplary recipient or host cells include Sp2/0 myeloma which normally does not produce endogenous immunoglobulin and mouse myeloma NS/0 cells. When transfected, the cell produces only immunoglobulin encoded by the transfected gene constructs. Transfected myelomas can be grown in culture or in the peritoneum of mice where secreted immunocytokine can be recovered from ascites fluid. Other lymphoid cells such as B lymphocytes can be used as recipient cells. Non-lymphoid recipient cells can be used as well, such as Chinese hamster ovary cells.

There are several methods for transfecting lymphoid cells with vectors containing the nucleic acid constructs encoding the chimeric immunoglobulin chain. For example, vectors may be introduced into lymphoid cells by spheroblast fusion (see, for example, Gillies et al. (1989) Biotechnol. 7: 798-804). Other useful methods include electroporation or calcium phosphate precipitation (see, for example, Sambrook et al. eds (1989) “Molecular Cloning: A Laboratory Manual,” Cold Spring Harbor Press).

Other useful methods of producing the immunocytokines include the preparation of an RNA sequence encoding the construct and its translation in an appropriate in vivo or in vitro expression system. It is contemplated that the recombinant DNA methodologies for synthesizing genes encoding antibody-cytokine fusion proteins, for introducing the genes into host cells, for expressing the genes in the host, and for harvesting the resulting fusion protein are well known and thoroughly documented in the art. Specific protocols are described, for example, in Sambrook et al. eds (1989) “Molecular Cloning: A Laboratory Manual,” Cold Spring Harbor Press.

It is understood that the chemically coupled immunocytokines may be produced using a variety of methods well known to those skilled in the art. For example, the antibody or an antibody fragment may be chemically coupled to the cytokine using chemically reactive amino acid side chains in the antibody or antibody fragment and the cytokine. The amino acid side chains may be covalently linked, for example, via disulfide bonds, or by means of homo- or hetero-bifunctional crosslinking reagents including, for example, N-succinimidyl 3(-2-pyridyylditio)propionate, m-maleimidobenzoyl-N-hydroxysuccinate ester, n7-maleimidobenzoyl-N-hydroxysulfosuccinimide ester, and 1,4-di-[3′(2′-pyridylthio)propionamido]butane, all of which are available commercially from Pierce, Rockford, Ill.

The compositions used in accordance with the methods of the present invention may be provided to an mammal by any suitable means, directly (e.g., locally, as by injection, implantation or topical administration to a tissue locus) or systemically (e.g., parenterally or orally). Where the composition is to be provided parenterally, such as by intravenous, subcutaneous, ophthalmic, intraperitoneal, intramuscular, buccal, rectal, vaginal, intraorbital, intracerebral, intracranial, intraspinal, intraventricular, intrathecal, intracistemal, intracapsular, intranasal or by aerosol administration, the composition preferably comprises part of an aqueous or physiologically compatible fluid suspension or solution. Formulations will be recognized and/or routinely developed by those skilled in the art.

Preferred dosages of the immunocytokine per administration are less than the maximum tolerated dose. For example, the immunocytokine may be administered at a dose less than half, less than a third, less than a quarter, or less than one-tenth of the maximum tolerated dose. The maximum tolerated dose (MTD) is the highest dose at which a substance can be administered at an acceptable toxicity level. Substances that are administered at the maximum tolerated dose, while at an acceptable level of toxicity, may still produce unpleasant side effects. The side effects produced by immunocytokines are much less severe than the side effects produced by cytokines alone. However, undesirable side effects have been reported with the use of various immunocytokines, including fever, nausea, vascular leakage and hypotension. The methods of the present invention allow for effective use of immunocytokines at a dose lower than the maximum tolerated dose, which has the advantage of producing fewer side effects. Although the dose to be administered will vary based on the immunocytokine used and other clinical parameters, in certain embodiments the dose is between 0.01 mg/kg and 20 mg/kg of immunocytokine, for example, at least 0.03-15 mg/kg, 0.03-6 mg/kg, 0.03-1.5 mg/kg, 0.03-0.6 mg/kg, 0.5-20 mg/kg, 0.5-15 mg/kg, 0.5-10 mg/kg, 0.5-6 mg/kg, 0.5-5 mg/kg, or 0.5-1.5 mg/kg. The dose of immunocytokine administered may vary throughout treatment. For example, a cytokine may be administered at a lower dose more frequently for a number of days, weeks, or months, and later be administered at a higher dose less frequently for a number of days, weeks, or months. For example, a dose of about 0.0375-0.6 may be administered 3 days a week for 3 consecutive weeks, followed by an increase in dose to about 0.0375-1.5 mg/kg administered one day a week for the following three weeks.

Administration of the immunocytokine may be by periodic bolus injections, or by continuous intravenous or intraperitoneal administration from an external reservoir (for example, from an intravenous bag) or internal (for example, from a bioerodable implant). It is contemplated, however, that the optimal combination of immunocytokines and radiation, modes of administration, and dosages may be determined by routine experimentation well within the level of skill in the art.

Radiation can kill immune effector cells. For example, T cells and dendritic cells in an irradiated tumor decrease immediately after irradiation. However, T-cell levels then rebound higher than baseline levels. Therefore, immunocytokine dose and dosing schedule in relation to the timing of radiation therapy is important. It is preferred that dosing is scheduled so that the rebound of immune effector cells following radiation therapy coincides with the immunostimulatory effects of administration of an immunocytokine.

In one embodiment, an immunocytokine is administered during radiation therapy. In a preferred embodiment, an immunocytokine is administered after radiation therapy. Administration of an immunocytokine during or, preferably, after radiation therapy results in a synergistic anti-tumor effect. Administration of an immunocytokine before radiation treatment may fail to produce the synergistic anti-tumor effect seen when immunocytokines are administered after radiation treatment. Similarly, extending radiation therapy for more than five days following the initiation of immunocytokine administration, may reduce the synergistic effect is seen than when administration of immunocytokines occurs after the end of radiation therapy. Without wishing to be bound by theory, the immunostimulatory effects of immunocytokine administration may be blunted by the initial immunosuppressive effects of radiation when radiation therapy is given after, or in some cases during, immunocytokine administration. On the other hand, immunocytokine administration should not occur so far after radiation therapy as to diminish the synergistic effect.

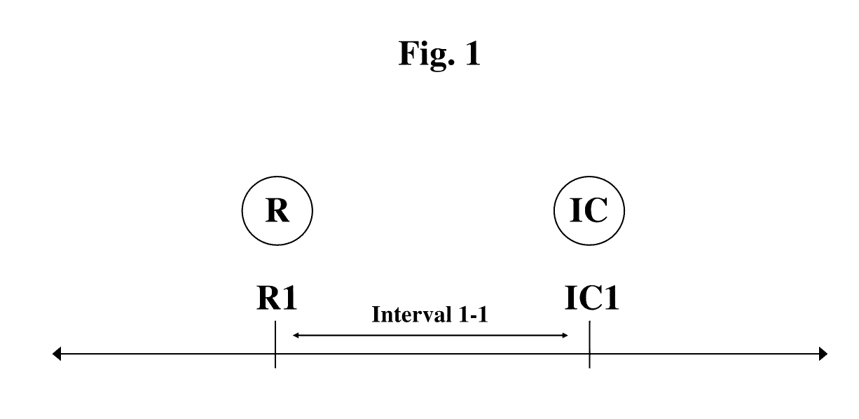

The radiation and the immunocytokine can each be administered as single doses, as depicted in the exemplary dosing schedule shown in FIG. 1. Referring to FIG. 1, radiation “R” is administered as a single dose on day “R1.” Following interval 1-1, which may be measured in hours or days, immunocytokine “IC” is administered as a single dose on day “IC1.” Preferably, the interval (interval 1-1) between the administration of radiation and immunocytokine is between 0-21 days. For example, the interval (interval 1-1) can be from 1-21 days, or from 2-21 days, or from 2-14 days, etc. Thereafter, a single dose of the immunocytokine may be administered at suitable intervals, for example, every 2 weeks, every 3 weeks, every 4 weeks, every 5 weeks, every 6 weeks, every 7 weeks, every 8 weeks, every 9 weeks, every 10 weeks, every 11 weeks, every 12 weeks, etc. An exemplary dosing schedule may be as follows. On day 1, a single dose of radiation is administered, and on day 4, immunocytokine treatment cycles begin, wherein the immunocytokine is administered as a single dose at 3-week intervals. After a suitable number of treatment cycles, for example 5 to 10, the dosing interval is increased, for example to up to 12 week intervals.

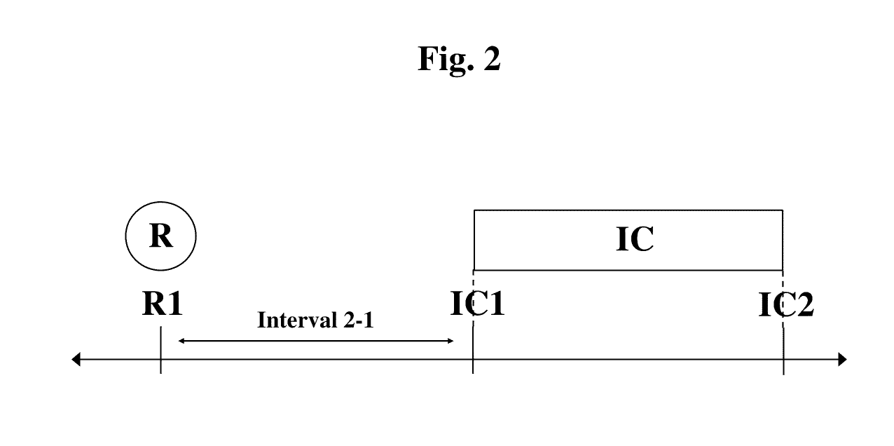

In another embodiment, as shown in FIG. 2, radiation is administered on only one day, followed by the administration of an immunocytokine on multiple days. Referring to FIG. 2, radiation “R” is administered as a single dose on day “R1.” Following interval 2-1, which may be measured in hours or days, administration of immunocytokine “IC” begins on day “IC1.” The immunocytokine may be administered daily, on alternate days, every third day, biweekly, weekly, continuously, or on any other suitable schedule, until administration terminates on day “IC2.” For example, the immunocytokine may be administered once daily on 2 consecutive days, 3 consecutive days, 4 consecutive days, etc. Preferably, the interval (interval 2-1) between administration of radiation and the start of administration of an immunocytokine is 0-21 days (e.g. between 1-21 days, between 2-21 days, between 2-14 days, etc.). Thereafter, the immunocytokine may be administered at suitable intervals, for example, every 2 weeks, every 3 weeks, every 4 weeks, every 5 weeks, every 6 weeks, every 7 weeks, every 8 weeks, every 9 weeks, every 10 weeks, every 11 weeks, every 12 weeks, etc. An exemplary dosing schedule may be as follows: On day 1 a single dose of radiation is administered, and on day 4 immunocytokine treatment cycles begin, wherein the immunocytokine is administered once daily on 3 consecutive days at 3-week intervals. After a suitable number of treatment cycles, for example 5 to 10, the dosing interval is increased, for example to up to 12 week intervals.

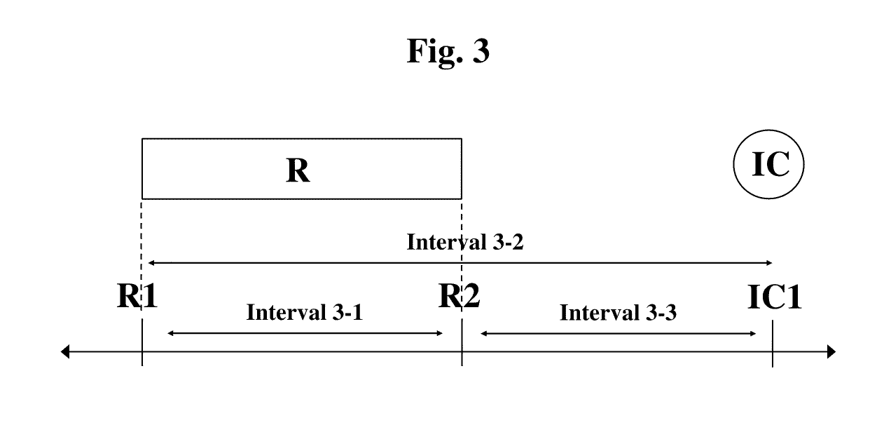

In yet another embodiment, as shown in FIG. 3, radiation “R” is administered over multiple days, followed by the administration of an immunocytokine “IC” on only one day “IC1.” Radiation is preferably administered over a period (interval 3-1) not exceeding 14 days (e.g. 4-10 days). Radiation may be administered daily, on alternate days, every third day, continuously, or on any other suitable schedule, until administration terminates on day “R2.” For example, the radiation may be administered on 3 consecutive days, on 4 consecutive days, on 5 consecutive days, etc. Administration of the immunocytokine can occur at least 5 days after the start “R1” of radiation therapy (interval 3-2), for example, 5 days after, 6 days after, etc. The interval (interval 3-3) between the end “R2” of radiation therapy and the administration of the immunocytokine can be 0 or more days, for example 1 day, 2 days, etc. Preferably, the interval (interval 3-3) between the end “R2” of radiation therapy and the administration of the immunocytokine “IC1” is at least 2 days, for example, 2 days, 3 days, etc. Thereafter, a single dose of the immunocytokine may be administered at suitable intervals, for example, every 2 weeks, every 3 weeks, every 4 weeks, every 5 weeks, every 6 weeks, every 7 weeks, every 8 weeks, every 9 weeks, every 10 weeks, every 11 weeks, every 12 weeks, etc. An exemplary dosing schedule may be as follows. On days −7 to −3 a fractionated dose of radiation is administered on 5 consecutive days (days −7, −6, −5, −4, and −3), and on day 1 (three days later) immunocytokine treatment cycles begin, wherein the immunocytokine is administered as a single dose at 3-week intervals. After a suitable number of treatment cycles, for example 5 to 10, the dosing interval is increased, for example to up to 12 week intervals.

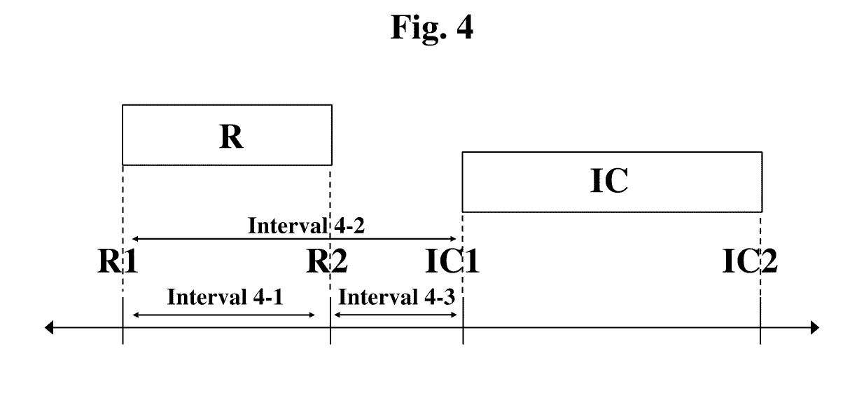

In another embodiment, as shown in FIG. 4, radiation “R” can be administered on more than one day, followed by administration of an immunocytokine “IC” on more than one day. Preferably, radiation is administered over a period (interval 4-1) not exceeding 14 days (e.g. 4-10 days). Radiation may be administered daily, on alternate days, every third day, continuously, or on any other suitable schedule, until administration terminates on day “R2.” For example, the radiation may be administered on 3 consecutive days, on 4 consecutive days, on 5 consecutive days, etc. The start of administration of the immunocytokine can occur at least 5 days after the start of radiation therapy (interval 4-2), for example, 5 days after, 6 days after, etc. The start of administration of the immunocytokine can occur at least 1 day after the end of radiation therapy (interval 4-3). Preferably the interval (interval 4-3) between the end “R2” of radiation therapy and the start of administration of the immunocytokine “IC1” is at least 2 days, for example, 2 days, 3 days, etc. The immunocytokine may be administered daily, on alternate days, every third day, biweekly, weekly, continuously, or on any other suitable schedule, until administration terminates on day “IC2.” For example, the immunocytokine may be administered once daily on 2 consecutive days, 3 consecutive days, 4 consecutive days, etc. Thereafter, the immunocytokine may be administered at suitable intervals, for example, every 2 weeks, every 3 weeks, every 4 weeks, every 5 weeks, every 6 weeks, every 7 weeks, every 8 weeks, every 9 weeks, every 10 weeks, every 11 weeks, every 12 weeks, etc. An exemplary dosing schedule may be as follows: On days −7 to −3 a fractionated dose of radiation is administered on 5 consecutive days, and on day 1 immunocytokine treatment cycles begin, wherein the immunocytokine is administered once daily on 3 consecutive days at 3-week intervals. After a suitable number of treatment cycles, for example 5 to 10, the dosing interval is increased, for example to up to 12 week intervals.

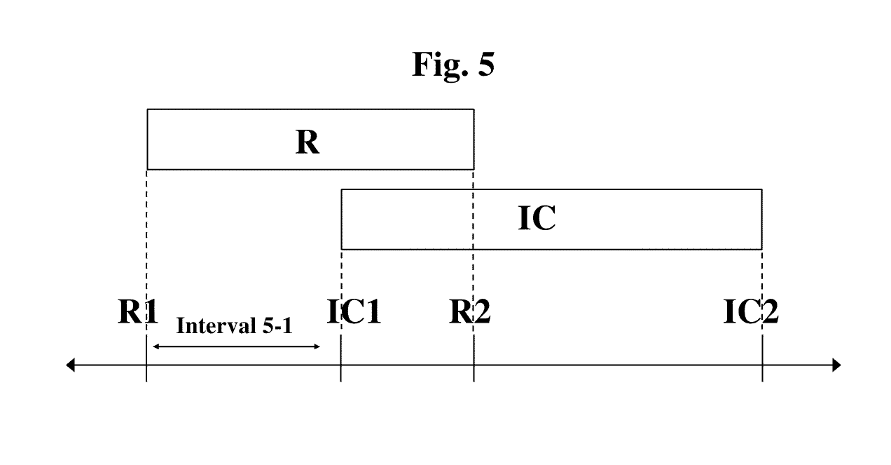

In another embodiment, as shown in FIG. 5, radiation “R” can be administered on more than one day, followed by administration of an immunocytokine “IC” on more than one day. Radiation may be administered daily, on alternate days, every third day, continuously, or on any other suitable schedule, until administration terminates on day R2. The interval (interval 5-1) between the start “R1” of administration of radiation and the start “IC1” of administration of an immunocytokine preferably does not exceed 14 days (e.g. 4-10 days). The start “IC1” of immunocytokine administration can occur before the end “R2” of the period of radiation administration. The immunocytokine may be administered daily, on alternate days, every third day, biweekly, weekly, continuously, or on any other suitable schedule, until administration terminates on day IC2.

While these embodiments describe a single treatment comprising a course of radiation followed by administration of the immunocytokine, it is envisaged that this treatment may be repeated as appropriate. For example, in a second round of treatment, the radiation may be administered to the same or to a different tumor site and/or the immunocytokine or a different immunocytokine may be subsequently administered.

Combination with Other Treatments

The inventive method can be performed in combination with other therapeutic agents or methods to achieve a desired biological effect in a patient. In one embodiment, the pharmaceutical composition is administered before, during, or after surgical resection of a tumor. Complete surgical removal of tumor tissue is often complicated by invasion of the tumor tissue into surrounding tissues and indefinite margins of the mass. As described herein, treatment of a tumor using the inventive method leads to tumor shrinkage, which will facilitate resection. Moreover, post-surgical performance of the inventive method can eliminate residual tumor cells.

Like surgical resection, chemotherapy is a standard treatment for most cancer types. Accordingly, the inventive method can be performed in parallel, before, or after chemical-based therapies. Common chemotherapeutics include, but are not limited to, adriamycin, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, floxuridine, fludarabine, fluorouracil, gemcitabine, hydroxyurea, idarubicin, ifosfamide, irinotecan, lomustine, mechlorethamine, mercaptopurine, meplhalan, methotrexate, mitomycin, mitotane, mitoxantrone, nitrosurea, paclitaxel, pamidronate, pentostatin, plicamycin, procarbazine, streptozocin, teniposide, thioguanine, thiotepa, vinblastine, vincristine, vinorelbine, taxol, transplatinum, 5-fluorouracil, and the like.

The inventive method can be performed alongside hormone therapy, which is the manipulation of hormone levels in the body to treat disease. Many cancers are somehow affected by the levels of hormones in the body and, as such, typical therapeutics associated with hormone therapy, e.g., tamoxifen, work to reduce the level circulating hormones and/or interrupt the binding of hormones to hormone receptors.

The inventive method can be performed in combination with administration of monoclonal antibodies, for example, bevacizumab (Avastin), cetuximab (Erbitux), gemtuzumab (Mylotarg), ibritumomab (Zevalin), matuzumab, and rituximab (Rituxin). Monoclonal antibodies act through a variety of mechanisms. For example, monoclonal antibodies can bind to specific tumor proteins and attract immune cells to the site of the tumor. Some monoclonal antibodies function by blocking growth signals that would otherwise allow tumors to grow and spread. Other monoclonal antibodies are attached to radioactive particles or chemotherapeutic drugs and act to deliver the radiation or drugs specifically to cancer cells.

### Target Tissue

Types of tumors and cancer cells to be treated with methods of the present invention include all types of solid tumors, such as those which are associated with the following types of cancers: lung, squamous cell carcinoma of the head and neck (SCCHN), pancreatic, colon, rectal, esophageal, prostate, breast, ovarian carcinoma, renal carcinoma, lymphoma and melanoma. The tumor can be associated with cancers of (i.e., located in) the oral cavity and pharynx, the digestive system, the respiratory system, bones and joints (e.g., bony metastases), soft tissue, the skin (e.g., melanoma), breast, the genital system, the urinary system, the eye and orbit, the brain and nervous system (e.g., glioma), or the endocrine system (e.g., thyroid) and is not necessarily the primary tumor. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non-small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system.

The tumor can be at any stage, and can be subject to other therapies. The inventive method is useful in treating tumors (i.e., destruction of tumor cells or reduction in tumor size) that have been proven to be resistant to other forms of cancer therapy. The tumor also can be of any size. Ideally, in treating the human for cancer, the inventive method results in a decreased rate of tumor growth, cancerous (tumor) cell death and/or reduction in tumor size. It will be appreciated that tumor cell death can occur without a substantial decrease in tumor size due to, for instance, the presence of supporting cells, vascularization, fibrous matrices, etc. Accordingly, while reduction in tumor size is preferred, it is not required in the treatment of cancer.

Preferably, the inventive method reduces the size of a tumor at least about 5% (e.g., at least about 10%, 15%, 20%, or 25%). More preferably, tumor size is reduced at about 30% (e.g., at least about 35%, 40%, 45%, 50%, 55%, 60% or 65%). Even more preferably, tumor size is reduced at least about 70% (e.g., at least about 75%, 80%, 85%, 90%, or 95%). Most preferably, the tumor is completely eliminated. However, as discussed herein, reduction of tumor size, although preferred, is not required. All that is required is the reduction in rate of growth of the tumor. For example, the tumor can slow its rate of growth, stop growing completely, shrink, or be completely eliminated. Any reduction in the rate of tumor growth is sufficient to realize a therapeutic effect.

### Assessment of Efficacy

Efficacy of the methods of the present invention can be measured in several ways. For example, measurements of tumor size, recurrence, survival, and initiation of immune response (e.g., using gene, proteomic, or cellular profiling) can be used to determine the efficacy of treatment. Measurements can be made before, during and after treatment to monitor the effectiveness of the methods of the present invention. The effectiveness of the methods can be monitored throughout the course of treatment for patients. Alternatively, the animal models described in the Examples section below or other suitable animal model can be used by a skilled artisan to test which combinations of immunocytokines and radiation and, optionally, other cancer treatments are most effective in acting synergistically to enhance the immune destruction of established tumors or cancer cells. Further, as novel immunocytokines are identified, a skilled artisan will be able to use these measures of efficacy to assess the potential of novel immunocytokines to enhance the anti-cancer effects of radiation.

Recurrence, survival and tumor size can be monitored to evaluate the efficacy of the methods of the present invention. Recurrence and survival can be assessed using statistical analyses known in the art. Tumor size in human patients can be monitored using a number of imaging methods known in the art, including endoscopy, radiographic imaging (including x-rays), Computed Tomography (CT) scans, Magnetic Resonance Imaging (MRI), and nuclear imaging. The effect of the therapy on tumor growth in animal models can be monitored by imaging methods, or by caliper or volumetric measurement.

Efficacy of the methods of the present invention can also be determined by measuring whether an immune response has been stimulated. Initiation of an immune response can be measured using a variety of methods. In one example, gene expression profiling can be performed to determine whether genes important to immune function have been stimulated by the methods of the present invention. Expression profiling can be performed on tumor samples or on other samples, such as lymph node samples or peripheral blood mononuclear cells, whose expression profiles can be indicative of the presence or absence of a systemic immune response. Samples can be taken from the patient before, during, and/or after treatment. RNA from the tumor tissue of treated mammals is extracted and the expression levels of genes associated with an immune response can be assessed, such as by quantitative real time PCR, before and after treatment with the present methods: RNA transcribed from genes involved in immune function is amplified to determine whether expression is increased or decreased throughout the course of treatment.

In another example, following therapy, tumors can be biopsied or excised, sectioned and stained via standard histological methods, or via specific immunohistological reagents in order to assess the effect of the combined therapy on immune response. For example, simple staining with hematoxolin and eosin can reveal differences in lymphocytic infiltration into the solid tumors which is indicative of a cellular immune response. Furthermore, immunostaining of sections with antibodies to specific classes of immune cells can reveal the nature of an induced response. For example, antibodies that bind to CD45 (a general leukocyte marker), CD4 and CD8 (for T cell subclass identification), CD25 (a lymphocyte marker) and NK1.1 (a marker on NK cells) can be used to assess the type of immune response that has been mediated by the methods of the invention. Additionally, antibodies against the immunocytokine used in the therapy can be employed to measure the extent to which the immunocytokine infiltrated the tumor.

The type and extent of immune response stimulated in response to the methods of the present invention also can be evaluated using fluorescence-activated cell sorting (FACS) to analyze immune effector cell populations. Using methods well-known in the art, processed samples, such as blood samples are exposed to a cocktail of antibodies against various immune cell markers, such as CD4, CD8, CD25, CD44, CD62L, CD11b, and DX5. Each species of antibody is labeled with a fluorescent label having a distinct emission wavelength from that of the other species of antibodies in the cocktail. Samples are then processed by a FACS sorter, such as the FacsARIA (BD Biosciences) and analyzed for various cell subpopulations.

Initiation of an immune response can also be measured using an Enzyme-linked immunosorbent spot (ELISPOT) assay. To determine whether a given immune cell has been stimulated, antibodies to a protein, for example, a cytokine, that is produced upon activation of the cell, are coated onto an ELISPOT plate. Splenocytes from mice treated with the methods of the present invention are added to the plate and incubated. If cells are activated, they will secrete the protein of interest, and the protein will be captured by the antibodies. Antibodies specific to a different epitope of the protein are added to the plate. These antibodies can be labeled with any one of a number of labels known in the art. Commonly, antibodies are biotin labeled, and streptavidin-HRP (horseradish peroxidase) is also added. Presence and amount of label is then measured to determine the extent to which an cell has been activated in the immune response.

Alternatively, the type of immune response mediated by the methods of the present invention can be assessed by conventional cell subset depletion studies described, for example, in Lode et al. (1998) Blood 91: 1706-1715. Examples of depleting antibodies include those that react with T cell markers CD4 and CD8, as well as those that bind the NK markers NK1.1 and asialo GM. Briefly, these antibodies are injected to the mammal prior to initiating the present methods at fairly high doses (for example, at a dose of about 0.5 mg/mouse), and are given at weekly intervals thereafter until the completion of the experiment. This technique can identify the cell-types necessary to elicit the observed immune response in the mammal.

In another approach, the cytotoxic activity of splenocytes isolated from animals having been treated with the combination therapy can be compared with those from the other treatment groups. Splenocyte cultures are prepared by mechanical mincing of recovered, sterile spleens by standard techniques found in most immunology laboratory manuals. See, for example, Coligan et al. (eds) (1988) “Current Protocols in Immunology,” John Wiley & Sons, Inc. The resulting cells then are cultured in a suitable cell culture medium (for example, DMEM from GIBCO) containing serum, antibiotics and a low concentration of IL-2 (˜10 U/mL). For example, in order to compare NK activity, 3 days of culture normally is optimal, whereas, in order to compare T cell cytotoxic activity, 5 days of culture normally is optimal. Cytotoxic activity can be measured by radioactively labeling tumor target cells (for example, LLC cells) with ⁵¹Cr for 30 min. Following removal of excess radiolabel, the labeled cells are mixed with varying concentrations of cultured spleen cells for 4 hr. At the end of the incubation, the ⁵¹Cr released from the cells is measured by a gamma counter which is then used to quantitate the extent of cell lysis induced by the immune cells. Traditional cytotoxic T lymphocyte (or CTL) activity is measured in this way.

### Authorization of Treatment or Payment for Treatment

According to the methods of the present invention, a third party, e.g., a hospital, clinic, a government entity, reimbursing party, insurance company (e.g., a health insurance company), HMO, third-party payor, or other entity which pays for, or reimburses medical expenses may authorize treatment, authorize payment for treatment, or authorize reimbursement of the costs of treatment. For example, the present invention relates to a healthcare method that includes authorizing the administration of, or authorizing payment or reimbursement for the administration of, an immunocytokine to a mammal with a tumor or cancer cells that were previously irradiated. For example, the healthcare method can include authorizing the administration of, or authorizing payment or reimbursement for the administration of, an immunocytokine to a mammal with a tumor previously irradiated at a dose of at least 1 Gy per day (e.g. at least 2, at least 3, 1-4, 1-10, 1-20, 2-4, 2-10, 2-20, 3-4, 3-10, or 3-20 Gy/day). The healthcare method of the present invention can include authorizing the administration of, or authorizing payment or reimbursement for the administration of, an immunocytokine when the immunocytokine is to be administered within 21 days (e.g. within 18, within 15, within 12, or within 8 days) of the first administration of radiation to the tumor. In one embodiment, the health care method can include authorizing the administration of, or authorizing payment or reimbursement for the administration of, an immunocytokine following radiation treatment, in which the immunocytokine is administered at a dose less than the maximum tolerated dose of the immunocytokine, for example, at a dose less than half, less than a third, less than a quarter, or less than one-tenth of the maximum tolerated dose of the immunocytokine.

### EXAMPLES

### Example 1

### Materials and Methods

### Proteins

NHS-IL2LT, also referred to as Selectikine or EMD521873, was produced from an NS/0 cell line and purified. NHS-muIL12 was produced from an NS/0 cell line and purified.

### Cells

CT26 cells, a murine colon epithelial cell line derived by intrarectal injection of N-nitroso-N-methylurethane in BALB/C mice, were transfected to express the human KS antigen (KSA or EpCAM) that was cloned by PCR and expressed in parental cells using a retroviral vector (Gillies 1998). CT26/KSA cells were maintained in DMEM, supplemented with 10% heat inactivated fetal bovine serum, L-glutamine, vitamins, sodium pyruvate, non-essential amino acids, penicillin/streptomycin and Geneticin® (G418) (Life Technologies, Inc.) at 37° C. and 7% CO₂. G418 was added to maintain KSA expression. CT26 and CT26/KSA cells were implanted in female BALB/C mice.

LL/2 (LLC) cells, a murine Lewis lung carcinoma cell line, were maintained in DMEM, supplemented with 10% heat-inactivated fetal bovine serum, L-glutamine, penicillin/streptomycin (Life Technologies, Inc.) at 37° C. and 7% CO2. LLC cells were implanted in female C57BL/6 mice.

B16 cells, a murine melanoma cell line, were maintained in RPMI 1640, supplemented with 10% heat inactivated fetal bovine serum, L-glutamine, sodium pyruvate, non-essential amino acids, penicillin/streptomycin (Life Technologies, Inc.) at 37° C. and 7% CO₂. B16 cells were implanted in female C57BL/6 mice.

### Chemicals and Solutions

EMD521873 was formulated in 128 mM arginine, 6 mM citrate, 2.35% sucrose, 0.05% Tween 80, pH 6.0. The protein concentrations of diluted solutions were determined using the absorbance at 280 nm and the theoretical extinction coefficient of 12.38 mg/OD₂₈₀, based on the known protein sequence. The stock solution was stored at 4° C. for less than 1 month. For dosing mice, an aliquot of formulated material was removed from stock vials, diluted with 0.9% saline, and injected into the animal within one hour after diluting. Unused diluted material was discarded.

NHS-muIL12 was formulated in 50 mM sodium phosphate, 150 mM sodium chloride, 0.05% Tween 80, pH 7.0. For dosing mice, the formulated material was diluted with 0.9% saline to 0.5 mg/ml.

Cisplatin (cis-diammineplatinum (II) dichloride) powder was obtained from Sigma-Aldrich® (catalog #P4394). A dosing solution of 0.4 mg/ml was prepared in 0.9% saline.

### Methods

For the tumor growth assay, cells growing exponentially in culture were injected as a single cell suspension in 100 μl of PBS intramuscularly into the upper legs of the mice. In experiments using a dual tumor model, cells were also implanted in the flanks of the mice. After tumors had become established, treatment was initiated (day 0). In Experiment LLC-7, tumors were implanted in the flank until established. Mice were treated on days 0 to 9, then, on day 12, tumors were excised using survival surgery. Thereafter, animal survival was monitored. In Experiment LLC-14, tumors were implanted subcutaneously in the flank until established. Mice were treated on days 0 to 6 and day 17, and tumor size was measured twice weekly for the duration of the study.

The following table summarizes the conditions for each experiment:

TABLE 1

| | Tumor | # cells | # days before | Initial tumor |
|---|---|---|---|---|
| Expt # | model | implanted IM | treatment | volume (mm3) |
| CT26-1 | CT26/SKA | 5 × 10⁵ | 12 | 193 |
| CT26-2 | CT26/SKA | 5 × 10⁵ | 11 | 199 |
| CT26-3 | CT26/SKA | 5 × 10⁵ | 12 | 127 |
| CT26-4 | CT26 | 5 × 10⁵ | 9 | 190 |
| B16-1 | B16 | 2 × 10⁵ | 9 | 97 |
| LLC-1 | LLC | 1 × 10⁵ | 7 | 109 |
| LLC-2 | LLC | 1 × 10⁵ | 7 | 110 |
| LLC-3 | LLC | 1 × 10⁵ | 10 | 139 |
| LLC-4 | LLC | 1 × 10⁵ | 7 | 106 |
| LLC-5 | LLC | 1 × 10⁵ | 8 | 184 |
| LLC-6 | LLC | 1 × 10⁵ leg | 8 | 96 |
| (leg) | | | | |
| LLC-6 | LLC | 2 × 10⁶ flank | 8 | 78 |
| (flank) | | | | |
| LLC-7 | LLC | 2 × 10⁶ flank | 8 | 130 |
| LLC-8 | LLC | 1 × 10⁵ | 7 | 106 |
| LLC-9 | LLC | 1 × 10⁵ | 7 | 119 |
| LLC-10 | LLC | 1.5 × 10⁵ | 9 | 132 |
| LLC-11 | LLC | 1 × 10⁵ | 9 | 110 |
| LLC-12 | LLC | 1 × 10⁵ | 8 | 115 |
| LLC-13 | LLC | 1 × 10⁵ | 7 | 121 |
| LLC-14 | LLC | 5 × 10⁵ flank | 10 | 135 |

Tumors were measured with calipers in three dimensions for the duration of the experiment. Tumor volumes were calculated using the equation:

Volume=4/3π(L/2×W/2×H/2)
where L=length, W=width and H=height of the tumor

Animals were weighed and general health was monitored during the course of the assay.

For local irradiation, mice were anesthetized by intraperitoneal injection of a solution of 2% tribromoethanol/3% xylazine. The amount injected was 0.02 ml/gram of the recipient's weight. Mice were restrained so that only their tumor-bearing leg was exposed to radiation, while the reminder of the mouse was shielded. Tumors were irradiated with ¹³⁷Cs gamma radiation at a dose rate of 1 Gy/min using a Gammacell® 40 Exactor (Nordion, Ottawa, ON).

Immune effector cell populations in blood were analyzed by FACS. Blood was collected from the retro-orbital sinus of each mouse. Red blood cells were eliminated using lysis buffer. After blocking with rat IgG (1:50), samples were incubated with a 7-color cocktail of antibodies against CD4, CD8, CD25, CD44, CD62L, CD11b, and DX5. Samples were run on a FacsARIA and analyzed for various cell subpopulations.

Enzyme-linked immunospot C (ELISPOT) assays for IFNγ induction in splenocytes were conducted by first coating ELISPOT plates with an anti-IFNγ antibody (murine IFNγ kit, BD Biosciences). Splenocytes from treated mice were added to the plates and incubated with an AH1:A5 peptide to stimulate IFNγ release from CT26-specific T-cells. Biotinylated IFNγ detection Ab and streptavidin-HRP were added to the plates. Resultant spots from individual IFNγ-producing lymphocytes were counted using a Zeiss® KS-ELISPOT plate reader.

Gene profiling of tumors was analyzed using excised tumor tissues placed in RNAlater® RNA stabilizing reagent upon harvest. Total RNA was prepared with an RNeasy® kit (Qiagen) and cDNA was prepared with the Superscript® III kit for qPCR (Life Technologies, Inc.). qPCR reactions were performed on an ABI7500 instrument in a standardized fashion and relative changes in gene expression were analyzed using the ddCt method (efficiency factor 95%). Housekeeping genes used for comparison included ACTB, B2M and HPRT1.

### Methods of Evaluation and Statistics

Data from the subcutaneous tumor growth assay are presented in graphic form depicting average and standard deviations (sd) of tumor volumes or weights during and after dosing. Animal weight changes were determined by subtracting the animal weight at each time point from the initial body weight, on day 0, then dividing by the initial body weight and multiplying by 100. Therefore a value of 0 indicates no weight change.

Student's 2-tailed t test or Mann-Whitney Rank Sum test, as appropriate, was performed on the individual tumor volumes or animal weights, to determine significant differences between treatment groups.

### Example 2

### Effect of a Single Dose of Radiation Followed by Immunocytokine Treatment

The effect of a single dose of radiation given on day 0 followed by i.v. dosing of the immunocytokine (5 mg/kg) was evaluated in three subcutaneous syngeneic tumor models (CT26 colon carcinoma, CT26/KSA colon carcinoma which expresses human EpCAM, and B16 melanoma). This example and, except where otherwise indicated, the following examples, used the exemplary immunocytokine dI-NHS76γ2(h)(FN>AQ)-ala-IL2(D20T), also designated as Selectikine or EMD521873 and described, for example, in U.S. Pat. No. 7,186,804.

In three separate experiments using the CT26/KSA model (Experiments CT26-1-3), irradiation of tumors with either 3 or 4 Gy followed by EMD521873 on days 2, 3, and 4 resulted in a strong synergistic effect in which a majority of the animals achieved complete regressions. ELISPOT analysis using an endogenous CT26 antigen demonstrated a >4-fold increase in T cell response compared to either therapy alone. A 4-fold increase in effector T cells in the blood was observed compared to either therapy alone while qPCR profiling techniques showed dramatic increases T cell and T cell activation-associated genes in the tumor with CD8, Granzyme B, and IFNγ gene expression increasing by at least 3.5-4.6 fold compared to either EMD521873 or radiation alone. The CD4⁺CD25⁺ lymphocyte subpopulation, which is associated with regulatory T cells, increased in the blood with the combination; however, the levels of FoxP3 gene expression in the tumor did not increase. In addition, when the immunocytokine KS-IL12 was tested under the same experimental conditions, the immune response measured within the tumor was very similar, indicating that the synergistic effects of administering radiation and immunocytokines are not limited to use with EMD521873, or even to immunocytokines with an NHS moiety or an IL2 moiety.

In the CT26 model (Experiment CT26-4), tumors were irradiated with either 4 or 8 Gy followed by i.v. EMD521873 on days 2, 3, and 4. The combination of EMD521873 with the 8 Gy dose blocked tumor growth beyond either therapy alone. Immune response in the tumor was not monitored.

In the B16 model (Experiment B16-1), irradiation of tumors with 10 Gy on d0 followed by i.v. EMD521873 on days 3, 4, and 5 resulted in greater anti-tumor activity compared to either treatment alone. In addition, there was a moderate, but significant increase in certain immune response markers in the tumor as measured by immune marker gene expression profiles with CD25, TNFA, TYROBP, ICOS and CD45 being modulated >4-fold compared to either therapy alone.

### Example 3

### Effect of Fractionated Dose of Radiation Followed by Multiple Immunocytokine Doses

The anti-tumor effects of five daily doses of 3.6 Gy given on days 0-4 followed by i.v. dosing of the exemplary immunocytokine EMD521873 (5 mg/kg) on days 7, 8, and 9 was evaluated in the Lewis lung carcinoma model (Experiments LLC-1, LLC-2). No complete responses were observed in the control or monotherapy groups, whereas 3/6 animals achieved complete responses with the combination. Gene expression profiling using a panel of immune markers demonstrated increases in markers for T cell, T cell activation, lymphocyte trafficking, and Th1 response for the combination compared to either radiation or EMD521873 alone. Genes that were upregulated include CD45, CISH, CD122, MGP, FASL, CD80, PTPRB, CD6, CCR7, TXK, CTLA4, PDCD1, IL10R, CCL6, CD8A, EOMES, CD28, TYROBP, ICAM1, CD206, VCAM1, CD3G, ITGAL, ITGB2, LAT, GZMK, STAT4, IL1A, CD115, MDM2, CD26, GIMAP3, CXCR4, LCK, HS6ST2. Genes downregulated include IL23A, SELE, SC4MOL, LDLR, SQLE, RAE1, CXCL1, CCL2.

### Example 4

### Effect of Adding Chemotherapy to the Radiation Plus Cytokine Combination

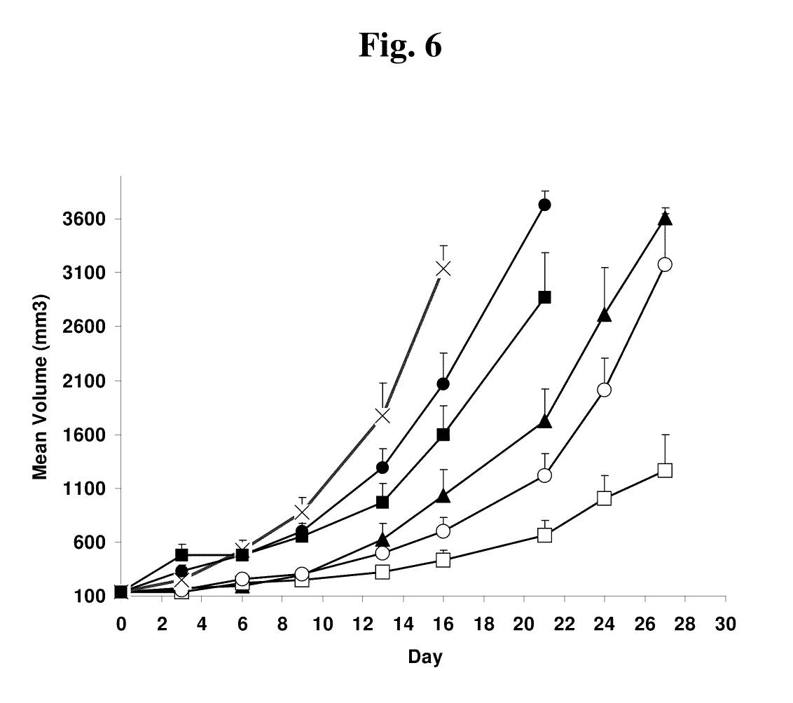

The anti-tumor effects of treating with immunocytokine (EMD521873, in this example) following radiation combined with chemotherapy treatment (chemoradiation) were evaluated in the Lewis lung subcutaneous tumor model (LLC-14). In this example, cisplatin was used as an exemplary chemotherapeutic agent. Tumor-bearing animals (n=10) were treated with either chemoradiation alone (cisplatin (4 mg/ml) and fractionated radiation (3.6 Gy/d, d0-4)), with EMD521873 (5 mg/kg/day i.v.) or NHS-muIL12 (10 μg/animal/day s.c.) alone, or with chemoradiation combined with the immunocytokine EMD521873 (5 mg/kg/day i.v.) or NHS-muIL12 (10 μg/animal/day s.c.). The immunocytokine was administered on only one day on d6 and again on only one day on d17. Tumor size was measured twice weekly until d24. As seen in FIG. 6, tumor growth of animals treated with chemoradiation combined with NHS-muIL12 (open squares) was significantly decreased compared to animals treated with chemoradiation alone (closed triangles), animals treated with NHS-muIL12 alone (closed squares), animals treated with EMD521873 alone (closed circles), or animals treated with saline (X). Chemoradiation also enhanced the efficacy of EMD521873 (open circles).

In another experiment (Experiments LLC-1, LLC-2, LLC-4), tumor-bearing animals were treated with either fractionated radiation alone (3.6 Gy/d, d0-4), cisplatin alone (4 mg/kg, d0), EMD521873 alone (5 mg/kg, d7, 8, 9), the dual combinations, or the triple combination. The combination of EMD521873 with radiation reduced tumor growth compared to any of the single treatments and to the other double treatments, and resulted in 3/6 complete regressions. The addition of cisplatin to the radiation/EMD521873 therapy resulted in further growth control and 5/6 complete regressions. These results demonstrate that therapy with either radiation or chemoradiation followed by EMD521873 improves the immune response and tumor control compared to radiation, chemoradiation, or EMD521873 therapy alone.

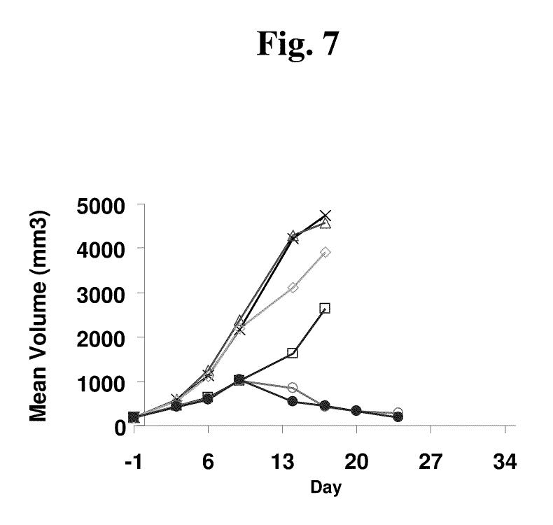

Similar results were seen when NHS-muIL12 was used as the immunocytokine under identical experimental conditions. As seen in FIG. 7, when NHS-muIL12 (closed circles) or EMD521873 (open circles) was administered with chemoradiation under the above-described experimental conditions, a decrease in tumor growth resulted compared to that seen using no treatment (X), using chemoradiation alone (open squares), using EMD521873 alone (open diamonds) or NHS-muIL12 alone (open triangles).

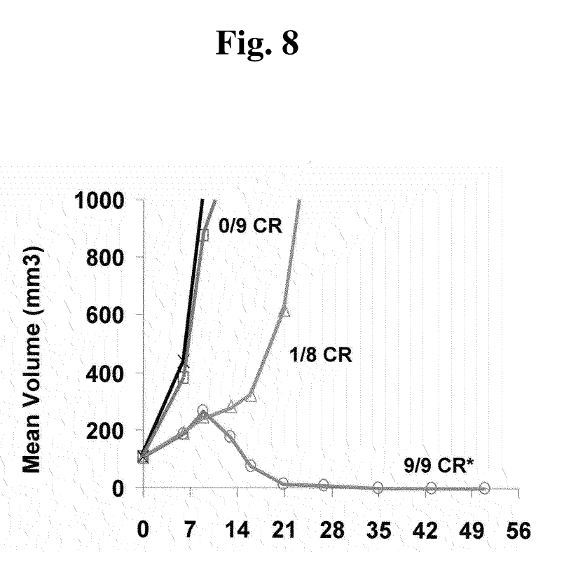

In another experiment the same experimental conditions were used, and the triple combination of chemoradiation plus EMD521873 (open circles) led to a decrease in tumor volume followed by 9/9 complete regressions. When chemoradiation alone (open triangles) was administered, on average no decrease in tumor growth was seen and only 1/9 complete regressions occurred. When a control treatment (X) or EMD521873 alone (open boxes) was used, no decrease in tumor growth was seen and no complete regressions occurred (FIG. 8).

### Example 5

### Ability of Local Irradiation Plus Immunocytokine Therapy to Generate a Systemic Anti-Tumor Response (Experiments LLC-3-LLC-7)

The ability of irradiation followed by intravenous administration of the exemplary immunocytokine EMD521873 to generate a long lasting immune response was tested by rechallenging cured mice with tumor cells. Mice bearing Lewis Lung Carcinoma tumors were originally treated with either fractionated radiation (3.6 Gy/d, d0-4) plus cisplatin (4 mg/kg, d0), EMD521873 alone (5 mg/kg, d7, 8, 9), or the combination of EMD521873 plus chemoradiation. The number of complete responses was determined and then those mice that had achieved complete remissions for greater than 50 days were rechallenged by s.c. injection of LLC cells followed by monitoring of tumor growth compared to naïve mice that had not previously been treated. Table 2 shows that 12 out of 13 mice that had achieved complete response rates with the EMD521873/Chemoradiation treatment did not regrow tumor, demonstrating that they had developed long term protective immunity to LLC tumor cells.

TABLE 2
Rechallenge of cured C57BL/6 mice with Lewis Lung Carcinoma cells

| | Complete Response Rate | | | | Immunity to Rechallenge | | | |
|---|---|---|---|---|---|---|---|---|
| Treat- | | | | Sel + | Naïve | | | Sel + |
| ment | Ctrl | Sel | Ch/Rx | Ch/Rx | Ctrl | Sel | Ch/Rx | Ch/Rx |
| Exp | 0/6 | 0/6 | 0/6 | 3/6 | 0/5 | n.a. | n.a. | 3/3 |
| LLC-3 | | | | | | | | |
| Exp | 0/8 | 1/8 | 1/8 | 7/8 | 0/5 | 0/1 | 1/1 | 5/6 |
| LLC-4 | | | | | | | | |
| Exp | 0/6 | 0/6 | 0/6 | 4/5 | 0/5 | n.a. | n.a. | 4/4 |
| LLC-5 | | | | | | | | |

In another experiment, LLC tumors were established subcutaneously in both the leg and flank of the same animal. Animals were then treated with either cisplatin (4 mg/kg, d0) plus fractionated radiation given to the leg tumor (3.6 Gy/d, d0-4), i.v. EMD521873 alone (5 mg/kg, d7, 8, 9), or the combination of EMD521873 plus chemoradiation (Experiment LLC-6). Tumor growth and immune response in both the irradiated leg tumor and the non-irradiated flank tumor were monitored. As in previous studies the combination of chemoradiation followed by EMD521873 achieved good growth control of the irradiated lesion. Although growth control of the flank tumor by the combination therapy did not surpass that of EMD521873 alone, analysis of the immune response for the combination therapy in the non-irradiated lesion demonstrated an enhancement of immune response. CD8 effector cells were increased by approximately 4-fold in the blood with the combination compared to either chemoradiation or EMD521873 alone. Immunohistochemical analysis showed that CD8 cell infiltration in tumors treated with the combination therapy group compared to the monotherapies was enhanced by 3.5-fold in the non-irradiated lesion versus 9-fold in the irradiated lesion. Immune gene expression profiling likewise showed in both leg tumors and flank tumors that EMD521873+chemoradiation increased CD3 (>20-fold, leg; 8-fold, flank), CD8 (10-fold, leg; 3.5-fold, flank), CISH (5.8-fold, leg; 3.2-fold, flank), CXCL9 (10.5-fold, leg; 14-fold, flank), and IFNg (14-fold, leg; 7-fold, flank) greater than EMD521873 or chemoradiation alone. Although the increases observed in the irradiated tumors were generally higher than those in the flank tumors, these results demonstrate that local radiation of a primary lesion plus chemotherapy and EMD521873 can generate a synergistic immune response in a tumor not in the field of radiation.

In another experiment the ability of local irradiation followed by immunocytokine administration to reduce tumor recurrence following surgery was tested (Experiment LLC-7). Mice with subcutaneous LLC tumors were treated with either chemoradiation (cisplatin 4 mg/kg, d0 plus 3.6 Gy/d, d0-4 of fractionated radiation given to the tumor) or EMD521873 (5 mg/kg, d7, 8, 9) plus chemoradiation. The remaining tumor was then surgically removed on day 12 or 13 and survival was monitored. Ten out of 12 mice treated with the combination achieved long term survival compared to 6 out of 10 treated with radiation alone. While the differences were not statistically significant (p=0.23), they did suggest that the combination would provide improved protection against recurrence or metastatic spread following surgery for the combination of EMD521873 plus chemoradiation compared to chemoradiation alone.

### Example 6

### Comparison of Treatment with Chemoradiation Plus Immunocytokine to Treatment with Chemoradiation Plus Cytokine (IL-2) Therapy (Experiments LLC-8 and LLC-9)

An experiment was designed to compare the combination of exemplary immunocytokine EMD521873 plus chemoradiation to exemplary cytokine IL-2 plus chemoradiation. Mice with subcutaneous LLC tumors were treated with either chemoradiation (cisplatin 4 mg/kg, d0 plus 3.6 Gy/d, d0-4 of fractionated radiation given to the tumor), i.v. EMD521873 alone (5 mg/kg, d7, 8, 9), i.v. IL-2 alone (equimolar IL-2 dose compared to EMD521873, 1.5 mg/kg, d7, 8, 9, 10, 11), or the combination of EMD521873 plus chemoradiation or IL-2 plus chemoradiation. Tumor growth control, response rate, and immune response within the tumor were then monitored.

For the EMD521873 combination, a total of 12 immune expression markers (GZMB, PDCD1, NKG7, NKG2D, CD3G, ITGAL, CD122, CD8A, FASL, CTLA4, INOS, CD25) were upregulated at least 3 to 6 times in the combination groups relative to the maximal level achieved in the monotherapies. In contrast, the effects of the combination of IL2 with chemoradiation in D10 tumor samples were similar to chemoradiation alone with no obvious contribution from the cytokine. Comparison of complete response rate and tumor growth control reflected the immune response data. The EMD521873 combination achieved better growth control and complete response rate (T/C=0.03 at day 21 with 4/6 animals with complete response) compared to the IL-2 combination (T/C=0.09 at day 21 with 2/6 animals with complete response). Importantly, the EMD521873 combination therapy achieved a superior memory response compared to the IL-2/chemoradiation combination. Four out of 4 of mice with complete responses from the EMD521873/chemoradiation combination were immune to rechallenge with LLC cells. In contrast neither of the 2 mice with complete responses in the IL-2/chemoradiation combination therapy were immune to rechallenge with tumor.

In addition, results similar to those seen with EMD521873/chemoradiation were seen with another immunocytokine, NHS-IL2 wt, in combination with chemoradiation under the same experimental conditions. NHS-IL2 wt is described in, for example, U.S. Pat. No. 7,186,804. This result demonstrates that the synergistic effect of radiation or chemoradiation and immunocytokine administration can be seen with immunocytokines other than EMD521873.

### Example 7

### Radiation Dose-Response in Combination with Fixed Dose and Schedule of Chemotherapy Plus Immunocytokine (Experiment LLC-3)

To examine how the dose of radiation affects the antitumor effect of the combination of chemoradiation plus immunocytokine, mice with subcutaneous LLC tumors were treated with cisplatin (4 mg/kg, d0) plus 0, 0.4, 1.2, or 3.6 Gy/d of local irradiation given on days 0-4. Chemoradiation was followed by either no treatment or EMD521873 (5 mg/kg, d7, 8, 9). Tumor size and response rate were then monitored. The EMD521873 combination with cisplatin plus 0.4 Gy/d did not improve tumor growth control to EMD521873 alone or EMD521873 plus cisplatin. The 1.2 Gy/d in combination with EMD521873 improved growth control compared to either therapy alone; however, no complete responses were achieved with either the combination or monotherapies. Finally, the cisplatin plus 3.6 Gy/d dose in combination with EMD521873 dramatically improved tumor growth control and complete response rate (T/C=0.02 at day 19 with 5/6 complete responses) compared to either the cisplatin plus 3.6 Gy/d group (T/C=0.32 at day 19 with 0/6 complete responses) or the EMD521873 alone group (T/C=0.69 at day 19 with 0/6 complete responses).

### Example 8

### Immunocytokine Dose Response in Combination with Fixed Dose and Schedule of Chemoradiation (Experiment LLC-10)

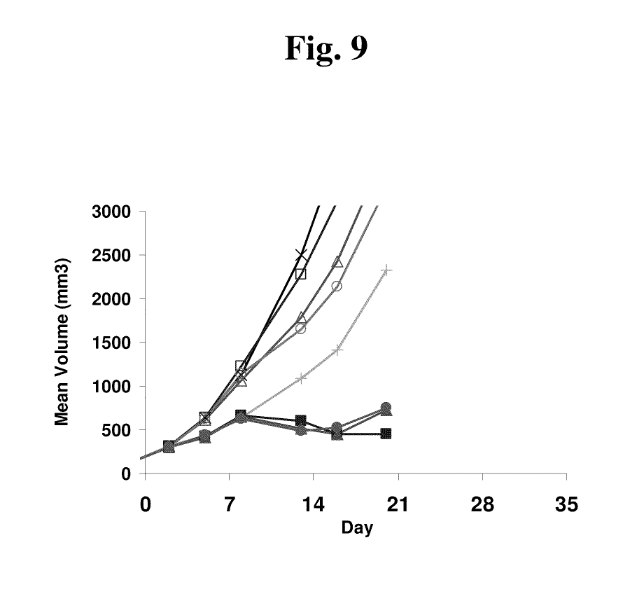

To examine how the dose of immunocytokine affects the antitumor effect of the chemoradiation plus immunocytokine combination, mice with subcutaneous LLC tumors were treated with increasing doses of i.v. EMD521873 (0, 1, 5, or 15 mg/kg, d7, 8, 9) either alone or in combination with chemoradiation (4 mg/kg cisplatin on d0 plus 3.6 Gy/d local tumor irradiation on d0-4). Tumor size, response rate, and immune gene modulation in the tumor were then monitored. No dose-response was established for the dose ranges used since all doses in combination with chemoradiation gave similar growth control, response rate (CRs=2/8, 3/8, and 3/8 for 1, 5, and 15 mg/kg EMD521873 plus chemoradiation, respectively), and immune gene modulation profiles. Effects on growth control are seen in FIG. 9: 1 mg/kg (closed square), 5 mg/kg (closed triangle) and 15 mg/kg (closed circle) EMD521873, in combination with chemoradiation, resulted in similarly reduced tumor volume compared to 1 mg/kg (open square), 5 mg/kg (open triangle) and 15 mg/kg (open circle) EMD521873 alone, chemoradiation alone (+), or vehicle alone (X). These results show that EMD521873 doses well below the maximum tolerated dose can be safely combined with chemoradiation without significantly affecting efficacy.

### Example 9

### Effect of Extending the Number of Radiation Doses Prior to Administering Immunocytokine (Experiment LLC-13)

To examine how extended dosing of radiation prior to dosing immunocytokine would impact the effectiveness of the combination, mice with subcutaneous LLC tumors were treated with chemoradiation for 1 week (cisplatin, 4 mg/kg on d0 and 3.6 Gy/d on d0, 2, 4), 2 weeks (cisplatin, 4 mg/kg on d0 and 3.6 Gy/d on d0, 2, 4, 7, 9, 11), or 3 weeks (cisplatin, 4 mg/kg on d0 and 3.6 Gy/d on d0, 2, 4, 7, 9, 11, 14, 16, 18) prior to giving the exemplary immunocytokine EMD521873 (administered in 3 daily i.v. doses 3 days after the last dose of radiation). Tumor size, response rate, and immune gene modulation in the tumor were then monitored.

Similar to previous results, mice treated with cisplatin plus radiation in the first week followed by EMD521873 achieved 4/10 complete responses and enhancement of tumor growth control compared to either therapy alone. In contrast, the mice treated with cisplatin plus radiation for two weeks or for three weeks prior to EMD521873 achieved only 2/10 or 0/10 complete responses, respectively, and neither gave enhancement of tumor growth control compared to chemoradiation alone. Furthermore, analysis of the immune response gene profiles in the tumors from mice treated with the different regimens indicated that radiation treatment in the first week followed by EMD521873 provided the strongest enhancement in key Th1-associated genes (approximate fold-inductions compared to untreated control: CD3, 50; CD8, 19; IFNγ, 22; CD25, 45; granzyme, 22; and perforin, 8). In comparison, the immune response was blunted when radiation was given in the 2 weeks prior to EMD521873 (approximate fold-inductions compared to untreated control: CD3, 14; CD8, 6; IFNγ, 10; CD25, 22; granzyme, 2; and perforin, 5) whereas giving radiation in the 3 weeks prior to EMD521873 resulted in a nearly complete abrogation of the immune response. These results demonstrate that giving chemoradiation for only one week prior to giving EMD521873 was superior to either the two week or three week radiation regimens.

### Example 10

### Effect of Administering Immunocytokine During Chemoradiation (Experiment LLC-12)

To examine how dosing immunocytokine during a course of radiation would impact the effectiveness of the combination, mice with subcutaneous LLC tumors were treated with radiation for 1 week (3.6 Gy/d; d0, 2, 4), 2 weeks (3.6 Gy/d; d0, 2, 4, 7, 9, 11), or 3 weeks (3.6 Gy/d; d0, 2, 4, 7, 9, 11, 14, 16, 18) with or without intravenous administration of the exemplary immunocytokine EMD521873 (5 mg/kg) on days 7, 8, 9. Tumor size, response rate, and immune gene modulation in the tumor were then monitored. Results showed that the administration of EMD521873 after a single week of radiation resulted in a majority of tumor regressions; however, there were no complete responses. Similarly, administration of EMD521873 during the 2^{nd} week of a 2 week course of radiation or during the 2^{nd} week of a 3 week course of radiation also resulted in tumor regressions and improved tumor growth control compared to EMD521873 or radiation alone. However, while dosing EMD521873 during the 2^{nd} week during a 3 week course of radiation did achieve additional tumor control, immune gene profiling demonstrated that the immune response in the tumor was blunted by the additional radiation compared to that observed in tumors receiving EMD521873 during the 2^{nd} week of either a 1 week or 2 week course of radiation. These immune effects are also in line with the results described above showing that continued radiation blunted the immune effects of the EMD521873/radiation combination.

### Example 11

### Effect of Administering Immunocytokine Prior to Chemoradiation (Experiment LLC-11)

To examine whether administering immunocytokine prior to radiation would result in enhanced anti-tumor effects, mice with subcutaneous LLC tumors were treated with the exemplary immunocytokine EMD521873 (5 mg/kg, d0, 1, 2) in the first week prior to treating with radiation in the 2^{nd} week (3.6 Gy/d; d7, 9, 11) or in the 2^{nd} and 3^{rd} weeks (3.6 Gy/d; d7, 9, 11, 14, 16, 18). Tumor size, response rate, and immune gene modulation in the tumor were then monitored. Results showed that the administration of EMD521873 followed by radiation for either 1 or 2 weeks did provide an increase in tumor growth control compared to EMD521873 or either radiation regimen alone. However, neither tumor regressions nor complete responses were achieved as was observed when radiation was given prior to EMD521873. Furthermore, analysis of the immune gene response profile for the combination was not significantly affected compared to EMD521873 or either radiation regimen alone. These results, together with the above results demonstrate that the timing and duration of the radiation relative to the EMD521873 dosing are important for achieving an anti-tumor immune response and tumor growth control.

### Example 12

### Treatment of Human Lung Cancer

An dose-escalation trial is performed using EMD521873 in combination with local irradiation (20 Gy) of primary tumors or metastases in subjects with non-small cell lung cancer stage IIIb with malignant pleural effusion or stage IV with disease control (partial response or stable disease) after application of 4 cycles of platinum-based, first-line chemotherapy.

Subjects receive local irradiation (5×4 Gy) given over 5 consecutive days, prior to the first treatment cycle with EMD521873. After a 2-day treatment-free interval, intravenous infusions of EMD521873 are given on 3 consecutive days in 3-week cycles.

In each cycle, patients receive EMD521873 given as a once-daily, 1-h intravenous infusion on 3 consecutive days (days 1-3) followed by an 18-day treatment-free break (days 4-21).

Dose escalation of EMD521873 is performed in cohorts of 3 subjects at dose levels of 0.15, 0.30, and 0.45 mg/kg. Escalation to the next dose level is based on the number of dose limiting toxicities (DLTs) observed, if any, during the DLT evaluation period (i.e. the 21 days following the day of first infusion of EMD528173 in the first cycle). DLTs are defined as any grade ≧3 toxicity assessed as related to trial treatment (EMD521873 and/or radiation). If no DLT occurs in the 3 subjects, the next 3 subjects are recruited at the next highest dose level. If 1 out of 3 subjects experiences DLT(s), an additional cohort of 3 subjects receive the same dose level. If 1 or more of the 3 additional subjects experience a DLT, the MTD will be deemed to have been exceeded and dose escalation will be stopped. The preceding dose level will be considered as the MTD. If 2 or 3 of the 3 subjects experience DLTs, the MTD will be deemed to have been exceeded, dose escalation will be stopped and the preceding dose level will be considered as the MTD.

If DLTs occur in 1 out of 6 subjects treated at the dose level of 0.15 mg/kg, an intermediate dose level of 0.225 mg/kg is introduced before escalating to 0.3 mg/kg. If DLTs occur in ≧2 subjects treated at the dose level of 0.15 mg/kg, the dose level of 0.075 mg/kg is explored. If 0 or 1 subject experiences a DLT at this dose level, 0.075 mg/kg would be considered the MTD.

It is anticipated that even subjects treated at a dose level below the MTD will demonstrate improved rates of tumor response, as determined by CT or MRI scan, and may demonstrate improvements in survival and/or lung function.

### Example 13

### Treatment of Human Melanoma

A study is performed to investigate the responses of cutaneous metastases in patients with stage IIIb-IV cutaneous/subcutaneous malignant melanoma to EMD521873 at the tumor site and in blood to EMD521873 in combination with local irradiation (25 Gy). The first treatment cycle with EMD521873 starts on day 1. Subsequent cycles are administered at 3-week intervals. Prior to the first dose of EMD521873, subjects are treated with two courses of local radiation (25 Gy) administered in fractions of 5 Gy on 5 consecutive days (days −28 to −24; and days −7 to −3) to different metastases.

For each patient, at least two cutaneous or subcutaneous lesions eligible for biopsy are selected for potential radiation (lesion A1 and lesion A2) and, if present, a third lesion (lesion A3) is also selected. A biopsy of a cutaneous/subcutaneous lesion (lesion A1) and blood sampling are performed at baseline (D-28). After 7 days, allowing for the necessary healing of the biopsy area, lesion A1 is irradiated (5×5 Gy) over 5 consecutive days starting on day −21 (D−21 to D−17). On day −7 (D−7) blood samples are withdrawn and a biopsy of the irradiated lesion A1 is taken. This tumor sample serves as an intraindividual control to assess the effect of radiation alone. On day −14, the pre-selected lesions A2 and A3 are also biopsied and blood drawn. Local radiation is performed only on lesion (A2) as above (5×5 Gy) but starting at day −7 (D−7 to D−3). After a 2 day treatment-free interval, i.e. starting on day 1, 0.3 mg/kg EMD521873 is administered as a 1 hour intravenous infusion on 3 consecutive days (D1-3). Blood sampling for immunomonitoring and biopsies of the irradiated lesion A2 and the non-irradiated lesion A3 are collected at the defined time points (D1 and D8 of cycle 1) for comparison to baseline (D-28) and to the intra-individual control (radiation only) and to the biopsies collected at D-14. It is anticipated that the non irradiated lesion (lesion A3) will evidence the systemic effect of the therapy on nonirradiated sites (abscopal effect).

Subjects receiving EMD521873 and radiation are expected to demonstrate reduced tumor progression (e.g. as defined by PET or CT scan) compared to control subjects receiving only EMD521873 in the absence of radiation treatment.

### INCORPORATION BY REFERENCE

The entire content of each patent and non-patent document disclosed herein is expressly incorporated herein by reference for all purposes.

### EQUIVALENTS

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are intended to be embraced therein.

What is claimed is:

1. A method of reducing tumor growth, the method comprising the step of:
(a) administering an immunocytokine to a mammal with a tumor previously irradiated at a dose of at least 1 Gy per day.

2. The method of claim 1, wherein the immunocytokine is administered within 21 days of an initial administration of radiation to the tumor.

3. The method of claim 2, further comprising repeating administration of the immunocytokine after 21 days of an initial administration of radiation to the tumor.

4. The method of claim 1, wherein the immunocytokine is administered at a dose less than the maximum tolerated dose of the immunocytokine.

5. The method of claim 1, wherein the mammal has cancer cells at multiple locations and wherein only a subset of the locations were previously irradiated.

6. The method of claim 1, wherein the immunocytokine is administered at least five days after an initial administration of radiation to the tumor.

7. The method of claim 1, wherein the tumor was previously irradiated on only one day.

8. The method of claim 1, wherein the tumor was previously irradiated on multiple days over a period not exceeding 14 days.

9. The method of claim 1, further comprising surgically removing at least a portion of the tumor.

10. The method of claim 1, wherein the tumor or cancer cells were irradiated with gamma irradiation.

11. The method of claim 1, wherein the tumor or cancer cells were irradiated at a dose of 3-20 Gy per day.

12. The method of claim 1, wherein the immunocytokine comprises interleukin-2.

13. The method of claim 12, wherein the interleukin-2 comprises a D20T mutation.

14. The method of claim 1, wherein the immunocytokine comprises interleukin-12.

15. The method of claim 1, further comprising administering an additional therapeutic agent.

16. The method of claim 1, the method further comprising the step, prior to step (a), of irradiating the tumor.

17. A method of reducing tumor growth, the method comprising the step of:
(a) administering an immunocytokine to a mammal with a previously irradiated tumor, wherein the immunocytokine is administered within 21 days of an initial administration of radiation to the tumor.

18. A method of reducing tumor growth, the method comprising the step of:
(a) administering an immunocytokine at a dose less than the maximum tolerated dose to a mammal with a previously irradiated tumor.

19. The method of claim 18, wherein the immunocytokine is administered at a dose less than half of the maximum tolerated dose.

20. The method of claim 19, wherein the immunocytokine is administered at a dose less than one-tenth of the maximum tolerated dose.

21. A method of enhancing a systemic immune response in a mammal having cancer cells at multiple locations, the method comprising the step of:
(a) administering an immunocytokine after a subset of the locations have been irradiated, wherein the radiation enhances an immune response both at irradiated and non-irradiated locations.

22. A method of reducing tumor growth, the method comprising the step of:
(a) administering an immunocytokine to a mammal with a tumor previously irradiated on multiple days over a period not exceeding 14 days.

23. The method of claim 22, wherein the period is 4-10 days.

24. The method of claim 22, wherein the immunocytokine is administered at least two days after the end of the period.
